(19) United States
(12) Patent Application Publication
(10) Pub. No.: US 2018/0243257 A1
(43) Pub. Date: Aug. 30, 2018

(54) COMBINATION OF FUROSEMIDE AND STEROIDS AND USES THEREFOR

(71) Applicant: Gregory LEVITIN, New York, NY (US)

(72) Inventor: Gregory LEVITIN, New York, NY (US)

(21) Appl. No.: 15/887,105

(22) Filed: Feb. 2, 2018

Related U.S. Application Data
(60) Provisional application No. 62/463,818, filed on Feb. 27, 2017.
(60) Provisional application No. 62/532,537, filed on Jul. 14, 2017.

(51) Int. Cl. A61K 31/341; A61K 31/573; A61K 9/08; A61K 9/00; A61P 11/00

(52) U.S. Cl. CPC A61K 31/341; A61K 31/573; A61K 9/0043; A61K 9/008; A61P 11/00; A61K 9/08

(57) ABSTRACT

A composition of furosemide, or a pharmaceutically acceptable salt, solvate, or physiologically functional derivative thereof is combined with a steroid, or a pharmaceutically acceptable salt, solvate, or physiologically functional derivative thereof, to provide a topical medication for nasal or pulmonary treatment. The composition can provide, in a single administration or dosing regime, the anti-inflammatory properties of the steroid and the anti-inflammatory properties of furosemide, without any significant interference between the two, or adverse reaction in situ.

### CROSS-REFERENCE TO RELATED APPLICATIONS

This application claims priority under 35 USC 119(e) of U.S. provisional applications Ser. Nos. 62/463,818, filed on Feb. 27, 2017, and 62/532,537, filed on Jul. 14, 2017, the disclosures of which are herein incorporated by reference.

### BACKGROUND OF THE INVENTION

The field of the invention pertains to a new pharmaceutical product or formulation for the treatment of respiratory tract diseases, including nasal polyposis and inflammatory airway diseases of the upper and lower respiratory tract.

Corticosteroids have been used orally in the past to treat all types of inflammatory conditions of the respiratory tract, but carry the potential for significant adverse side effects. Compared to oral therapy, the development of inhaled corticosteroids and their derivatives in aerosol form was notable for its high clinical activity but rapid metabolism to less active forms when absorbed, thus minimizing potential side effects such as hypo-thalamo-pituitary axis suppression and growth suppression.

Inflammatory diseases of the upper and lower respiratory tract include, but are not limited to: allergic, perennial, and vasomotor rhinitis; sinusitis, otitis media, nasal polyposis, asthma, bronchitis, chronic obstructive pulmonary disease, emphysema, cystic fibrosis, rhinovirus related illness, and pneumonia. Such conditions may also be referred to herein as nasal and pulmonary inflammatory diseases.

It is known to treat nasal and pulmonary inflammatory diseases using a corticosteroid, which will suppress nasal and pulmonary inflammatory conditions. The corticosteroids used in aerosol form within the respiratory tract that have been shown to have high topical clinical pharmacologic activity with little to no systemic effects include: beclomethasone, betamethasone, mometasone, budesonide, flunisolide, cyclosenide, and triamcinolone.

It is has also been described to use furosemide for the treatment of inflammatory-related conditions of the respiratory tract. Thus, for example, it is known to use furosemide as a nasal spray to treat certain specific nasal and pulmonary inflammatory diseases, such as allergic and non-allergic rhinitis and nasal polyposis. It is also known to use furosemide in aerosolized form to treat asthma. However there are no known formulations of furosemide in production, such that furosemide as either a nasal spray or aerosol must be made on an individual basis at a compounding pharmacy.

Combinations of furosemide treatments with steroid treatments may prove to be beneficial, however, because furosemide is not presently readily available on the market, no known clinical investigations have been conducted with respect to the efficacy of such combinations.

### SUMMARY OF THE INVENTION

As disclosed below, it has been found, surprisingly, that providing a treatment that combines the effects of steroid treatments and furosemide treatments, in a pharmaceutically acceptable formulation, which is tolerated in situ is highly desirable and can be done without significantly disrupting the potency of the constituent pharmaceuticals.

In one aspect of the invention, a composition of furosemide, or a pharmaceutically acceptable salt, solvate, or physiologically functional derivative thereof, can advantageously be combined with a steroid, or a pharmaceutically acceptable salt, solvate, or physiologically functional derivative thereof, to provide a stable, very effective topical medication for nasal or pulmonary treatment. The composition can provide, in a single administration or dosing regime, the anti-inflammatory properties of the steroid and the anti-inflammatory (and/or other) properties of furosemide, without any significant interference between the two, or adverse reaction in situ.

It is believed that this unique composition has:
a) the potential to decrease the daily dosage of corticosteroids to achieve the desired clinical response (e.g., the corticosteroid does is a lower dose than found in other comparable medications or with corticosteroids used alone)
b) an equal or greater clinical effect than corticosteroid therapy alone
c) the potential to decrease incidence of clinical exacerbations requiring oral corticosteroid therapy (e.g., less frequent exacerbations as compared to other modes of treatment)
d) the potential to achieve shorter duration of topical corticosteroid therapy to reach desired clinical effect (e.g., stabilize the disease)

The composition merges the preceding factors together to produce a topical medication, such that the medication combines intense pharmacologic activity with no or minimal systemic effects, yields equal or greater clinical efficacy, and delivers the corticosteroid at the lowest dose possible while maintaining clinical effectiveness.

In one aspect of the present invention, a pharmaceutical composition includes furosemide or a pharmaceutically acceptable salt, solvate, or physiologically functional derivative thereof, and a steroid or a pharmaceutically acceptable salt, solvate, or physiologically functional derivative thereof (hereinafter, the “composition”). In certain embodiments, the composition is a topical formulation, and certain topical formulations of the composition may be in a form suitable for nasal or pulmonary administration. In certain embodiments, the steroid may be a corticosteroid.

The present invention also relates to a method for treating, preventing, or reversing exacerbations of inflammatory diseases of the upper and lower respiratory tract in a patient, whether the patient is a child or adult includes administering to the patient a dose of a pharmaceutical composition which includes furosemide or a pharmaceutically acceptable salt, solvate, or physiologically functional derivative thereof, and a steroid or a pharmaceutically acceptable salt, solvate, or physiologically functional derivative thereof. In certain embodiments, the administered composition is a topical formulation, and certain topical formulations of the composition may be in a form suitable for nasal or pulmonary administration. In certain embodiments, the steroid in the administered composition may be a corticosteroid.

The term “physiologically functional derivative” as used herein denotes a chemical derivative of any of the specific therapeutic agents described herein having the same or similar physiological function as the free base therapeutic agent and, for example, being convertible in the body thereto. In certain embodiments, the physiologically functional derivatives include esters.

### DETAILED DESCRIPTION OF THE EMBODIMENTS

As used throughout, ranges are used as shorthand for describing all values within the stated range, up to and including the stated beginning and end values of the range. Each disclosed range describes each and every value that is within the range, such that any value within a disclosed range can be selected as a terminus for an alternative range. In addition, all references cited herein are hereby incorporated by referenced in their entireties. In the event of a conflict in a definition in the present disclosure and that of a cited reference, the present disclosure controls.

The composition includes furosemide or a pharmaceutically acceptable salt, solvate, or physiologically functional derivative thereof, and a steroid or a pharmaceutically acceptable salt, solvate, or physiologically functional derivative thereof. The composition may be formed into one or more different types of topical formulations, including, but not limited to, nasal drops, eye drops, nasal sprays, nasal inhalation solutions or aerosols or insufflation powders, and pulmonary inhalation solutions or aerosols or insufflation powders. The pharmacologic formulations of the composition are generally suitable for pulmonary, buccal, or nasal administration.

The composition may be an aqueous topical formulation, and the examples included below describe application of such a formulation. As described herein, the composition may include other types of formulations. Those of ordinary skill in the art will appreciate that certain formulations of the composition may be suitable for administration through other routes, including, but not limited to, oral, parenteral including subcutaneous, intradermal, intramuscular, intravenous and intraarticular, intranasal, inhalation (including fine particle dusts or mists which may be generated by means of various types of metered dose pressurized aerosols, nebulizers, or insufflators), rectal and topical (including dermal, buccal, sublingual and intraocular administration), with the understanding that the most suitable route may depend upon for example the condition and disorder of the recipient. The formulations of the composition may conveniently be presented in unit dosage form and may be prepared by methods known in the art of pharmacy. All methods include the step of bringing the active ingredients into association with the carrier, which constitutes one or more accessory ingredients/excipients. In general the formulations are prepared by uniformly and intimately bringing into association the active ingredient with liquid carriers or finely divided solid carriers or both and then, if necessary, shaping the product into the desired formulation.

The composition may include a stable aqueous solution of furosemide or one or more of its salts, in combination with steroids, which may be beclomethasone, mometasone, fluticasone, triamcinolone, budesonide or cyclosenide, such that the aqueous solution may be used in the form of an inhalation solution, pressurized aerosol, nasal drops, or in the form of a spray. The spray may be formed by the use of the aqueous solution in combination with a conventional dispenser, such as a spray-squeeze bottle or a pump vaporizer. In certain embodiments, a compressed gas aerosol dispenser may be used.

The amount of furosemide used in the composition may range between 10 μg to 100 μg. Exemplary doses of the amount of furosemide in the composition include 10, 20, 25, 30, 40, 50, 60, 70, 75, 80, 90, or 100 μg. In certain embodiments, the dose released in each individual actuation of a dispenser may be 25, 50, 75, or 100 μg. The amount of steroid used in the composition may range between 10 μg to 150 μg. Exemplary doses of the amount of steroid in the composition may include 20, 25, 30, 40, 50, 60, 75 or 100 μg of the steroid to be released per individual actuation of the dispenser. Different steroids may have different suitable dosages in the composition. The steroid included in the composition may be a corticosteroid, examples of which include beclomethasone, betamethasone, mometasone, budesonide, flunisolide, cyclosenide, and triamcinolone.

The dose of the composition is preferably administered at least once daily. For certain variants of the composition and/or depending upon the patient and/or patient diagnosis, the dose may be administered twice daily. A once daily dose may be administered any time of the day, e.g., in the morning or in the evening. The administration of a daily dose of the composition may be part of a continuous treatment regimen which lasts more than a single day (i.e., multiple doses are administered over a period of days). The treatment regimen, for certain patients, may last for a period of weeks, months, or years, depending upon the diagnosis and the determinations of the attending physician.

The patient may be a child or an adult. For purposes of this disclosure, a child refers to a human being between 2 to 17 years of age. For purposes of this disclosure, an adult refers to a human being 18 years of age or older.

In certain embodiments, administration of a daily dose of the composition results in effective treatment and or prophylaxis and or reversal of exacerbation of nasal polyposis and inflammatory respiratory diseases. Inflammatory airway diseases of the upper and lower respiratory tract can involve the nasal cavity or the pulmonary cavity.

Formulations of the composition containing a corticosteroid in combination with furosemide have use in the prophylaxis and treatment of clinical conditions for which a glucocorticoid and/or furosemide is indicated. Such conditions associated with the nasal cavity or upper respiratory tract include conditions associated with inflammation of the nasal cavity such as allergic and seasonal rhinitis, chronic and perennial rhinitis, vasomotor rhinitis, and atrophic rhinitis; and nasal polyposis which includes occurrences of single and multiple nasal polyps in either the left, right or bilateral sinonasal cavities.

Formulations of the composition containing a corticosteroid in combination with furosemide have use in the prophylaxis and treatment of clinical conditions for which a glucocorticoid and/or furosemide is indicated. Such conditions associated with the pulmonary or lower respiratory tract include conditions associated with inflammation of the lower respiratory tract such as reversible or reactive airway obstruction (e.g., asthma, nocturnal asthma, exercise-induced asthma); chronic obstructive pulmonary disease (COPD) (e.g., chronic and wheezy bronchitis, emphysema), and respiratory tract infections (e.g., bronchitis and pneumonia).

The composition may be used for the treatment of respiratory diseases including, but not limited to, nasal polyposis, allergic rhinitis, perennial or chronic rhinitis, perennial rhinitis, vasomotor rhinitis, bronchitis, and asthma.

In certain embodiments, the composition is provided in a form suitable for inhalation. Formulations for inhalation include powder compositions, which preferably contain lactose, and spray compositions which may be formulated, for example, as an aqueous solution or suspension or as an aerosol delivered from a pressurized pack, with the use of a suitable propellant such as 1,1,1,2-terafluorethane, 1,1,1,2,3,3,3-heptafluoropropane, carbon dioxide or other suitable gas. Other propellants suitable for delivery of pharmaceutical formulations as part of a pressurized aerosol are disclosed in EP 0372777, WO91/04011, WO91/11173, WO91/11495, WO91/14422, WO93/11743, and EP-0553298.

The formulations of the composition may be manufactured by preparing a drug concentrate of the active ingredients with additional solvents and or excipient materials to create a stable solution, and then placing the solution into an aerosol vial or canister suitable for the storage and delivery of a metered amount of the formulation.

The formulations of the composition may contain a preservative and/or stabilizer. These include, for example: ethylene diamine tetra-acetic acid (edetic acid) and its alkali salts (for example dialkali salts such as disodium salt, calcium salt, calcium-sodium salt), lower alkyl p-hydroxybenzoates, chlorhexidine (for example in the form of the acetate or gluconate) and phenyl mercury borate. Other suitable preservatives are: pharmaceutically useful quaternary ammonium compounds, for example cetylpyridinium chloride, tetradecyltrimethyl ammonium bromide, generally known as “cetrimide”, benzyldimethyl-[2-[2-[p-(1,1,3,3-tetramethyl-butyl)phenoxy] ethoxy]-ammonium chloride, generally known as “benzethonium chloride” and myristyl picolinium chloride. Each of these compounds may be used in a concentration of 0.002 to 0.05%, for example 0.02% (weight/volume in liquid formulations, otherwise weight/weight). Preservatives among the quaternary ammonium compounds may be alkylbenzyl dimethyl ammonium chloride and mixtures thereof, for example the compounds generally known as “benzalkonium chloride.”

The total amount of preservatives in the formulations (solutions, ointments, etc.) may be from 0.001 to 0.10 g. In certain embodiments the total amount of preservatives may be 0.01 g per 100 ml of solution/suspension or per 100 g of formulation.

In the case of preservatives, the following amounts of individual substances can, for example, be used: thimerosal: 0.002-0.02%; benzalkonium chloride: 0.002 to 0.02% (in combination with thimerosal the amount of thimerosal may be, for example 0.002 to 0.005%); chlorhexidine acetate or gluconate: 0.01 to 0.02%; phenyl mercuric/nitrate, borate, acetate: 0.002-0.004%; p-hydroxybenzoic acid ester (for example, a mixture of the methyl ester and propyl ester in the ratio 7:3): 0.05-0.15%, or alternatively 0.1%.

The preservative used may be a combination of edetic acid (for example, as the disodium salt) and benzalkonium chloride. In this combination, the edetic acid may be used in a concentration of 0.05 to 0.1%, benzalkonium chloride may be used in a concentration of 0.005 to 0.05%, or alternatively 0.01%.

In the case of solutions/suspensions reference is always made to percent by weight/volume, in the case of solid or semi-solid formulations to percent by weight/weight of the formulation.

Further auxiliary substances which may be used for the formulations of the composition include, but are not limited to: polyvinyl pyrrolidone, sorbitan fatty acid esters such as sorbitan trioleate, polyethoxylated sorbitan fatty acid esters (for example polyethoxylated sorbitan trioleate), sorbimacrogol oleate, synthetic amphotensides (tritons), ethylene oxide ethers of octylphenolformaldehyde condensation products, phosphatides such as lecithin, polyethoxylated fats, polyethoxylated oleotriglycerides and polyethoxylated fatty alcohols. In this context, polyethoxylated means that the relevant substances contain polyoxyethylene chains, the degree of polymerisation of which is generally between 2 to 40, in particular between 10 to 20. These substances may be used to improve the solubility of the furosemide component.

As an option, it is possible to use additional isotonization agents. Isotonization agents which may be used include, but are not limited to: saccharose, glucose, glycerine, sorbitol, 1,2-propylene glycol and NaCl. The isotonization agents adjust the osmotic pressure of the formulations to the same osmotic pressure as nasal secretion. For this purpose, these substances are in each case to be used in such amount that, in the case of a solution, a reduction in the freezing point of 0.50 to 0.56° C. may be attained in comparison to pure water.

It is also possible to add thickening agents to solutions of the composition to prevent the solution from flowing out of the nose too quickly and to give the solution a viscosity of about 1.5 to 3 mPa, or alternatively 2 mPa.

Such thickening agents may include, but are not limited to: cellulose derivatives (for example cellulose ether) in which the cellulose-hydroxy groups are partially etherified with lower unsaturated aliphatic alcohols and/or lower unsaturated aliphatic oxyalcohols (for example methyl cellulose, carboxymethyl cellulose, hydroxypropylmethylcellulose), gelatin, polyvinylpyrrolidone, tragacanth, ethoxose (water soluble binding and thickening agents on the basis of ethyl cellulose), alginic acid, polyvinyl alcohol, polyacrylic acid, pectin and equivalent agents. Should these substances contain acid groups, the corresponding physiologically acceptable salts may also be used.

In the event hydroxypropyl cellulose is used, 0.1% by weight of the formulation may be used for this purpose.

In the event Avicel RC 591 or CL11 is used, 0.65-3.0% by weight of the formulation may be used for this purpose.

It is also possible to add to the formulations buffer substances including, but not limited to: citric acid/sodium hydrogensulphate borate buffer, phosphates (sodium hydrogenorthophosphate, disodium hydrogenphosphate), trometamol or equivalent conventional buffers. Such buffer substances may be used to adjust the formulations to a pH value of 3 to 7, or alternatively 4.5 to 6.5.

The amount of citric acid is may be 0.01 to 0.14 g, or alternatively 0.04 to 0.05 g; the amount of disodium hydrogenphosphate may be 0.1 to 0.5 g, or alternatively 0.2 to 0.3 g, per 100 ml of solution. The weights given relate in each case to the anhydrous substances.

In the case of solutions and suspensions, the maximum total concentration of active agent and buffer may be less than 5%, or alternatively less than 2% (weight/volume).

For the nasal application, a solution or suspension may be used which is applied as an aerosol, i.e., in the form of a fine dispersion in air or in another conventional carrier gas, for example by means of a conventional pump vaporizer.

Application as a dosage aerosol is also possible. Dosage aerosols are defined as being pressure packings which contain the furosemide or its salts in combination with steroid, in the form of a solution or suspension in a so-called propellant. The propellant may be a pressurized liquid chlorinated, fluorinated hydrocarbon or mixtures of various chlorinated, fluorinated hydrocarbons as well as propane, butane, isobutene or mixtures of these among themselves or with chlorinated, fluorinated hydrocarbons which are gaseous at atmospheric pressure and room temperature. Hydrofluorocarbons (HFCs), such as HFC 134a, and HFC 227a can also be used, and are preferred for environmental reasons. The pressure packing has a dosage or metering valve which, on actuation, releases a defined amount of the solution or suspension of the medicament. The subsequent very sudden vaporization of the propellant tears the solution or suspension of furosemide into the finest droplets or minute particles which can be sprayed in the nose or which are available for inspiration into the nose. Certain plastic applicators may be used to actuate the valve and to convey the sprayed suspension into the nose.

In the case of application as an aerosol, it is also possible to use a conventional applicator.

Certain embodiments of the composition are hereinafter described and it will be appreciated that any of the previous description of suitable ingredients and formulation characteristics can also be applicable to the following products and formulations.

The composition may be formed as a pharmaceutical product including (i) furosemide, or a pharmaceutically acceptable salt, solvate, or physiologically functional derivative thereof, provided in an aerosol formulation together with a propellant typically suitable for MDI delivery, and (ii) at least one steroid, or a pharmaceutically acceptable salt, solvate, or physiologically functional derivative thereof, provided in an aerosol formulation together with a propellant typically suitable for MDI delivery. Such a pharmaceutical product may be prepared for simultaneous, separate or sequential use in the treatment of conditions for which administration of furosemide and/or one or more steroid is indicated.

The composition may also be formed as an aerosol formulation which is suitable for MDI delivery comprising (i) furosemide, or a pharmaceutically acceptable salt, solvate, or physiologically functional derivative thereof, and (ii) at least one steroid, or a pharmaceutically acceptable salt, solvate, or physiologically functional derivative thereof, together with a propellant.

The respective therapeutic agents of the aerosol formulation may be administered simultaneously, either in the same or different pharmaceutical formulations, or separately or sequentially. If there is separate or sequential administration, the subsequently administered therapeutic agents should be administered to a patient within a time scale so as to achieve, or more particularly optimize, the above advantageous synergistic therapeutic effect of the composition.

Suitable propellants for use in pharmaceutical products of formulations of the composition include, but are not limited to: 1,1,1,2-tetrafluoroethane (HFA 134a) or 1,1,1,2,3,3,3,-heptafluoropropane (HFA 227), or a combination of both, or mono-fluoro trichloromethane and dichloro difluoromethane, in particular 1,1,1,2-tetrafluoroethane (HFA 134a) or 1,1,1,2,3,3,3-heptafluoropropane (HFA 227), with HFA 134a being preferred.

A pharmaceutical aerosol formulation of the composition may further include a polar cosolvent such as C2-6 aliphatic alcohols and polyols, for example ethanol, isopropanol and propylene glycol, with ethanol often being preferred. In certain embodiments, the concentration of the cosolvent is in the range of about 2 to 10% by weight, typically up to about 5%, of the total formulation.

A pharmaceutical aerosol formulation of the composition may further include one or more surfactants. Such surfactants may be included to stabilize the formulations and for lubrication of a valve system. Some of the most commonly used surfactants in aerosol formulations are oils derived from natural sources, such as corn oil, olive oil, cottonseed oil and sunflower seed oil, and also phospholipids. Suitable surfactants may include lecithin, oleic acid or sorbitan oleate.

The composition may also be formulated as an insufflatable powder. In certain embodiments of such an insufflatable powder, the maximum particle size of the substance suitably does not exceed 10 μm. Furosemide or its salts and the steroid may be mixed with inert carrier substances or drawn up onto inert carrier substances. Carrier substances which may be used include, but are not limited to: sugars such as glucose, saccharose, lactose and fructose. Also starches or starch derivatives, oligosaccharides such as dextrins, cyclodextrins and their derivatives, polyvinylpyrrolidone, alginic acid, tylose, silicic acid, cellulose, cellulose derivatives (for example cellulose ether), sugar alcohols such as mannitol or sorbitol, calcium carbonate, calcium phosphate, etc.

In certain embodiments, the therapeutic agents employed may have a particle size of less than about 10 μm, and alternatively less than 5 μm.

The use of insufflation powders may result in the composition being a pharmaceutical product which includes (i) furosemide, or a pharmaceutically acceptable salt, solvate, or physiologically functional derivative thereof, provided as an insufflation powder, and (ii) at least one steroid, or a pharmaceutically acceptable salt, solvate, or physiologically functional derivative thereof, provided as an insufflation powder. Such a pharmaceutical product may be prepared for simultaneous, separate or sequential use in the treatment of conditions for which administration of furosemide and/or one or more steroid is indicated.

The respective therapeutic agents of the pharmaceutical product may be administered simultaneously, either in the same or different insufflation powder formulations, or separately or sequentially. If there is separate or sequential administration as discussed above, the subsequently administered therapeutic agents should be administered to a patient within a time scale so as to achieve, or more particularly optimize, the above advantageous synergistic therapeutic effect of the composition.

In certain embodiments, the composition may also be an insufflation powder formulation which includes (i) furosemide, or a pharmaceutically acceptable salt, solvate, or physiologically functional derivative thereof, and (ii) at least one steroid, or a pharmaceutically acceptable salt, solvate, or physiologically functional derivative thereof, together with a pharmaceutically acceptable carrier or excipient therefor.

Dry insufflation powder formulations may be beneficial where it is required that therapeutic agents are retained in the nasal cavity, and systemic side effects can be minimized or eliminated. Furthermore, insufflation powder formulations may be beneficial because retention of furosemide, or a pharmaceutically acceptable salt, solvate, or physiologically functional derivative thereof, at the nasal mucosa is improved, the bitter aftertaste associated with liquid formulations may be significantly reduced, while also exhibiting the above advantageous synergistic therapeutic effect of the composition. By providing a dry insufflation powder formulation of furosemide, together with a steroid, having an average particle size of less than about 10 μm, the therapeutic agents may be restricted primarily to the desired target organ, the nasal or pulmonary mucosa.

A dry powder insufflation formulation for the composition may be administered by the use of an insufflator, which can produce a finely divided cloud of the dry powder. The insufflator may include a mechanism for administration a substantially pre-determined amount of the formulation of the composition. The powder may be used directly with an insufflator which is provided with a bottle or container for the powder, or the powder may be filled into a capsule or cartridge, such as a gelatin capsule, or other single dose device adapted for administration. The insufflator may also include a mechanism for opening the capsule or other dose device.

Combinations of therapeutic agents employed in pharmaceutical products and formulations of the composition (in particular nasal sprays or drops, aerosol or insufflation products and formulations as described above) may include any one of the combinations described herein.

In certain embodiments, the composition may be a pharmaceutical product which includes (i) furosemide, or a pharmaceutically acceptable salt thereof, and (ii) at least one steroid selected from the group consisting of beclomethasone, fluticasone, mometasone and pharmaceutically acceptable esters thereof. Such a pharmaceutical product may be prepared for simultaneous, separate or sequential use in the treatment of conditions for which administration of furosemide and/or one or more steroid is indicated. Suitably the esters may be selected from beclomethasone dipropionate, fluticasone propionate, fluticasone valerate, mometasone furoate and mometasone furoate monohydrate.

In certain embodiments, the composition may be a pharmaceutical formulation which includes (i) furosemide, or a pharmaceutically acceptable salt thereof, and (ii) at least one steroid selected from the group consisting of beclomethasone, fluticasone, mometasone and pharmaceutically acceptable esters thereof, together with a pharmaceutically acceptable carrier or excipient therefor. The esters may be selected from beclomethasone dipropionate, fluticasone propionate, fluticasone valerate, mometasone furoate and mometasone furoate monohydrate.

In the case of a nasal spray, the formulation of the composition may include furosemide, or a pharmaceutically acceptable salt thereof, together with mometasone either as the free base or in ester form, such as mometasone furoate.

In certain embodiments, the composition may be a pharmaceutical formulation which includes any one of the following combinations:
furosemide and beclomethasone dipropionate;
furosemide and fluticasone propionate;
furosemide and fluticasone valerate;
furosemide and mometasone furoate; and
furosemide and mometasone furoate monohydrate.

In another aspect, the invention is directed toward a method for the prophylaxis or treatment in a mammal, such as a human, of conditions for which administration of furosemide and/or one or more steroid is indicated. The method includes administration of a therapeutically effective amount of a composition formed as a pharmaceutical formulation substantially as hereinbefore described. Such a pharmaceutical formulation may be prepared for simultaneous, separate or sequential administration for the treatment of such conditions.

In another aspect, the invention is directed toward the manufacture of a medicament for the prophylaxis or treatment in a mammal, such as a human, of conditions for which administration of furosemide and/or one or more steroid is indicated. Such a pharmaceutical product may be prepared for simultaneous, separate or sequential use in the treatment of such conditions.

In another aspect, the invention is directed toward a process of preparing a pharmaceutical product substantially as hereinbefore described. This process includes providing as a combined preparation for simultaneous, separate or sequential use in the treatment of conditions for which administration of furosemide and/or one or more steroid is indicated: (i) furosemide, or a pharmaceutically acceptable salt, solvate, or physiologically functional derivative thereof, and (ii) at least one steroid, or a pharmaceutically acceptable salt, solvate, or physiologically functional derivative thereof.

In another aspect, the invention is directed toward a process of preparing a pharmaceutical formulation substantially as hereinbefore described. This process includes admixing a pharmaceutically acceptable carrier or excipient with: (i) furosemide, or a pharmaceutically acceptable salt, solvate, or physiologically functional derivative thereof, and (ii) at least one steroid, or a pharmaceutically acceptable salt, solvate, or physiologically functional derivative thereof. Such a pharmaceutical formulation may be formed as an insufflation powder formulation, a nasal spray, or a nasal or pulmonary inhalation solution or aerosol, substantially as hereinbefore described.

The following examples illustrate the efficacy of the composition described above. Such examples are not intended to limit the scope of the invention in any way. In Examples where only the ingredients of formulations of the composition are listed, these formulations are prepared by techniques well known in the art.

### EXAMPLES

The following patients with a diagnosis of nasal polyposis with at least more than one polyp were given simultaneous administration of the two compounds in separate formulations one immediately following the other on a regimented basis, where the patient was an adult over 18 years of age, the dosage was a single daily dose, the period of delivery was at least 30 consecutive days, and for some patients delivery at least 60 days, and the patients continued on this therapy for the entire period of follow up for up to 90 days. For the purposes of clinical measurement, nasal polyps were graded as a percentage of blockage of the middle meatus and sinonasal tracts at initial presentation and at each follow up visit.

### Example 1

A 34 year old male presented with clinical findings of extensive nasal polyposis causing near complete obstruction of his sinonasal tracts bilaterally. He had failed several months of topical nasal steroid therapy and had been advised to undergo functional sinus surgery and polypectomy but was instead prescribed a five day course of oral prednisone followed by a dosing regimen of daily corticosteroid and furosemide nasal aerosol therapy. His sinonasal passages were graded 100% blocked on the left and 100% blocked on the right.

At the 30 day follow up, he had noted significant improvement in nasal airflow and polyps were re-graded at 50% obstructed on left and 40% on the right.

At the 60 day follow up, he continued to improve and had less than 20% obstruction on the left and less than 10% obstruction on the right.

At the 90 day follow up, he had suffered an upper respiratory tract infection 2 weeks earlier. Despite this, his nasal passages remained stable with slight interval improvement with less than 10% obstruction on the right side and essentially clear on the left side.

### Example 2

A 43 year old male had bilateral nasal polyps causing over 60% blockage of the left side and 80% of the right side. He was prescribed a dosing regimen of daily corticosteroid and furosemide nasal aerosol therapy.

At the 30 day follow up, he had noted significant improvement in nasal airflow and polyps were re-graded at 25% obstructed on left and 35% on the right.

At the 90 day follow up, he had 20% obstruction on the left and less than 10% obstruction on the right.

### Example 3

A X38 year old woman had chronic nasal polyposis and known environmental allergies and had undergone functional sinus surgery and office nasal polypectomy twice in the last four years. Despite treatment with topical nasal steroid therapy throughout this four year period, she presented with recurrent nasal polyposis bilaterally and was graded with 50% obstruction on the left and 80% on the right. She was prescribed a dosing regimen of daily corticosteroid and furosemide nasal aerosol therapy

At the 30 day follow up, she had noted significant improvement in nasal airflow and polyps were re-graded at 30% obstructed on left and 60% on the right.

At the 60 day follow up, she had suffered an upper respiratory tract infection during the interval period and had less than 20% obstruction on the left and less than 30% obstruction on the right.

At the 90 day follow up, her nasal passages continued to improve with less than 10% obstruction on either side.

### Example 4

A 52 year old male was diagnosed with nasal polyposis and had been treated with several months of topical steroid therapy with persistent nasal polyps. He had 40% obstruction on the left and 60% obstruction on the right. He was prescribed a dosing regimen of daily corticosteroid and furosemide nasal aerosol therapy.

At the 30 day follow up, he had noted significant improvement in nasal airflow and polyps were re-graded at 50% obstructed on left and 50% on the right.

At the 60 day follow up, he continued to improve and had less than 20% obstruction on either side.

### Example 5

A 39 year old female had a history of chronic sinusitis and bilateral nasal polyposis and failed previous oral and topical steroid therapy. She presented with 80% obstruction on the left side and 80% obstruction on the right side. She was treated with a 5 day course of oral prednisone and then prescribed a dosing regimen of daily corticosteroid and furosemide nasal aerosol therapy.

At the 30 day follow up, she had noted significant improvement in nasal airflow and polyps were re-graded at 50% obstructed on left and 70% on the right.

At the 60 day follow up, she continued to improve and had less than 10% obstruction on the left and essentially clear on the right.

### Example 6

A 38 year old male with a history of allergic rhinitis, which was previously treated with intranasal steroid sprays, presented with a left nasal polyp blocking the left middle meatus by 50%; the right side was clear. He was prescribed a dosing regimen of furosemide and corticosteroid applied topically as a nasal spray.

At the 30 day follow up, he had noted significant improvement in nasal airflow and the polyp was re-graded at 30% obstructed on left.

At the 60 day follow up, he continued to improve and had less than 10% obstruction on the left and was asymptomatic.

At the 90 day follow up, he had suffered an upper respiratory tract infection 2 weeks earlier and had slight increase in polyps with approximately 20% obstruction on the left and 20% obstruction on the right. Despite this, his nasal passages remained clear and he required no oral steroid therapy.

While the invention has been described with respect to specific examples and modes of carrying out the invention, those skilled in the art will appreciate that there are numerous variations and permutations of the above described composition and processes. It is to be understood that other embodiments which have not been expressly disclosed herein may be utilized without departing from the scope of the present invention. Thus, the spirit and scope of the invention should be construed broadly as set forth in the appended claims.

What is claimed is:

1. A pharmaceutical composition comprising:
furosemide, or a pharmaceutical salt thereof; and
a steroid, or a pharmaceutical salt thereof.

2. The pharmaceutical composition according to claim 1, wherein the composition is in the form of a formulation selected from the group consisting of nasal drops, eye drops, nasal sprays, nasal inhalation solutions, aerosol powders, nasal insufflation powders, pulmonary inhalation solutions, pulmonary aerosols and pulmonary insufflation powders.

3. The pharmaceutical composition according to claim 1, wherein the formulation is in aqueous solution.

4. The pharmaceutical composition according to claim 1, wherein the steroid is selected from the group consisting of beclomethasone, mometasone, fluticasone, triamcinolone, budesonide and cyclosenide.

5. The pharmaceutical composition according to claim 1, wherein the furosemide is present in an amount between 10 μg and 100 μg per dose.

6. The pharmaceutical composition according to claim 1, wherein the steroid is present in an amount between 10 μg and 150 μg per dose.

7. The pharmaceutical composition according to claim 1, wherein the composition is in a form suitable for inhalation and contains a propellant.

8. The pharmaceutical composition according to claim 1, wherein the composition contains at least one of a preservative and a stabilizer.

9. The pharmaceutical composition according to claim 1, wherein the composition contains at least one of an isotonization agent, a surfactant, a polar cosolvent, a thickening agent and a buffer.

10. The composition according to claim 4, wherein the steroid is selected from the group consisting of beclomethasone dipropionate, fluticasone propionate, fluticasone valerate, mometasone furoate and mometasone furoate monohydrate.

11. A method for treating an inflammatory disease in an upper or lower respiratory tract of a mammal comprising administering a therapeutically effective amount of a furosemide and a steroid.

12. The method according to claim 11, wherein the furosemide and steroid are administered sequentially.

13. The method according to claim 11, wherein the furosemide and steroid are administered simultaneously.

14. The method according to claim 11, wherein the furosemide and steroid are administered intranasally.

15. The method according to claim 11, wherein the furosemide and steroid are administered by inhalation.

16. The method according to claim 11, wherein the furosemide and steroid are administered in a dosage of 10-100 μg of furosemide and 10-150 μg of steroid per dose.

17. The method according to claim 11, wherein the furosemide and steroid are mixed with a propellant and administered using an applicator having a pressure packing and a dosage or metering valve.

18. A method of preparing a pharmaceutical composition, comprising admixing a pharmaceutically acceptable carrier or excipient with:
furosemide or pharmaceutically acceptable salt, solvate or a physiologically function derivative thereof, and
a steroid or a pharmaceutically acceptable salt, solvate or physiologically functional derivative thereof.

19. The method according to claim 18, further comprising adding a propellant to the composition and placing the composition in a container configured for metered dosage of the composition.

20. The method according to claim 18 further comprising adding at least one of the following substances to the composition: an isotonization agent, a surfactant, a polar cosolvent, a thickening agent and a buffer.
